(19) United States
(12) Patent Application Publication
(10) Pub. No.: US 2026/0330657 A1
(43) Pub. Date: Oct. 22, 2026

(54) SYSTEMS, APPARATUSES, AND METHODS FOR ESTIMATING DIAPHRAGM MOTION DURING MAGNETIC RESONANCE IMAGING

(71) Applicant: Siemens Healthineers AG, Forchheim (DE)

(72) Inventors: Xinzhou Li, St. Louis, MO (US); Bernd Kühn, Uttenreuth (DE)

(21) Appl. No.: 19/185,895

(22) Filed: Apr. 22, 2025

(51) Int. Cl. G06T 7/277; A61B 5/00; G06T 5/70; G06T 7/00

(52) U.S. Cl. CPC G06T 7/277; A61B 5/7207; A61B 5/721; G06T 5/70; G06T 7/0012; G06T 2207/10088; G06T 2207/20081; G06T 2207/30004; G06T 2207/30168

(57) ABSTRACT

A system includes at least one memory configured to store instructions and at least one processor configured to execute the instructions to cause the system to acquire, during a first phase, first respiratory signals of a first type and first respiratory signals of a second type, determine a correlation model correlating the first respiratory signals of the first type and the first respiratory signals of the second type, update the correlation model based on second respiratory signals of the first type and second respiratory signals of the second type, acquired during a second phase, the second phase including an imaging acquisition period during which the second respiratory signals of the first type are acquired, and estimate diaphragm motion based on the second respiratory signals of the first type and the correlation model.

### TECHNICAL FIELD

The present disclosure relates to systems, apparatuses, methods, and/or non-transitory computer-readable storage mediums for estimating diaphragm motion during magnetic resonance (“MR”) imaging.

### BACKGROUND

Respiratory motion may impact quality of magnetic resonance (“MR”) imaging, especially for imaging of the thorax and the upper abdomen. In particular, motion artifacts may be present in images from MR imaging of regions impacted by respiratory motion. Methods of correcting motion artifacts may be used to address respiratory motion in MR imaging.

### SUMMARY

At least one example embodiment relates to a system that may include at least one memory configured to store instructions and at least one processor configured to execute the instructions to cause the system to acquire, during a first phase, first respiratory signals of a first type and first respiratory signals of a second type, determine a correlation model correlating the first respiratory signals of the first type and the first respiratory signals of the second type, update the correlation model based on second respiratory signals of the first type and second respiratory signals of the second type, acquired during a second phase, the second phase including an imaging acquisition period during which the second respiratory signals of the first type are acquired, and estimate diaphragm motion based on the second respiratory signals of the first type and the correlation model.

In at least one example embodiment, the at least one processor may be further configured to cause the system to process the diaphragm motion with a filter. In at least one example embodiment, the filter may be a Kalman filter and a procession model of the Kalman filter may be trained using a non-supervised learning method.

In at least one example embodiment, the correlation model may be a quadratic function defining a relationship between the first respiratory signals of the first type and the first respiratory signals of the second type.

In at least one example embodiment, the second respiratory signals of the second type may be generated from a navigator-triggered sequence and the second respiratory signals of the second type may not be acquired during the imaging acquisition period.

In at least one example embodiment, the first phase may be a training phase and the second phase may be an imaging phase.

In at least one example embodiment, the diaphragm motion may be an estimation of the second respiratory signals of the second type during the imaging acquisition period.

In at least one example embodiment, the at least one processor may be further configured to cause the system to correct for motion artifacts in an image obtained during the imaging acquisition period based on the diaphragm motion.

In at least one example embodiment, the at least one processor may be further configured to cause the system to predict an overall image quality before the imaging acquisition period is complete.

In at least one example embodiment, the first respiratory signals of the first type and the second respiratory signals of the first type may be acquired by one or more sensors, the one or more sensors may include at least one of respiratory bellows, biomatrix respiratory sensors, or a pilot tone.

Also described herein is a method. The method may include acquiring, during a first phase, first respiratory signals of a first type and first respiratory signals of a second type, determining a correlation model correlating the first respiratory signals of the first type and the first respiratory signals of the second type, updating the correlation model based on second respiratory signals of the first type and second respiratory signals of the second type, acquired during a second phase, the second phase including an imaging acquisition period during which the second respiratory signals of the first type are acquired, and estimating diaphragm motion based on the second respiratory signals of the first type and the correlation model.

In at least one example embodiment, the method may further include processing the diaphragm motion with a filter. In at least one example embodiment, the filter may be a Kalman filter and a procession model of the Kalman filter may be trained using a non-supervised learning method.

In at least one example embodiment, the correlation model may be a quadratic function defining a relationship between the first respiratory signals of the first type and the first respiratory signals of the second type.

In at least one example embodiment, the second respiratory signals of the second type may be generated from a navigator-triggered sequence and the second respiratory signals of the second type may not be acquired during the imaging acquisition period.

In at least one example embodiment, the first phase may be a training phase and the second phase may be an imaging phase.

In at least one example embodiment, the diaphragm motion may be an estimation of the second respiratory signals of the second type during the imaging acquisition period.

In at least one example embodiment, the method may further include correcting for motion artifacts in an image obtained during the imaging acquisition period based on the diaphragm motion.

In at least one example embodiment, the method may further include predicting an overall image quality before the imaging acquisition period is complete.

In at least one example embodiment, the first respiratory signals of the first type and the second respiratory signals of the first type may be acquired by one or more sensors, the one or more sensors may include at least one of respiratory bellows, biomatrix respiratory sensors, or a pilot tone.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1A is an illustration of a system for implementing methods according to example embodiments.

FIG. 1B is a block diagram illustrating an example embodiment of the system shown in FIG. 1A.

FIG. 2 is a diagram illustrating diaphragm motion in a learning phase and an imaging phase in accordance with at least one example embodiment.

FIG. 3 is a diagram of a surrogate signal of respiratory motion from a sensor and estimated diaphragm motion during an imaging phase in accordance with at least one example embodiment.

FIGS. 4A, 4B, 4C and 4D are diagrams of estimated diaphragm motion during an imaging phase in accordance with at least one example embodiment.

FIG. 5 illustrates a method for reducing motion artifacts from a resultant image based on estimated diaphragm motion in accordance with at least one example embodiment.

FIG. 6 illustrates a method for estimating diaphragm motion at S508, in accordance with at least one example embodiment.

FIG. 7 illustrates a method for predicting image quality based on estimated diaphragm motion in accordance with at least one example embodiment.

FIG. 8 is a diagram of a diaphragm position at a trigger point and estimated diaphragm motion during an imaging phase in accordance with at least one example embodiment.

FIG. 9 is an image acquired during the imaging phase of FIG. 8 in accordance with at least one example embodiment.

FIG. 10 is a diagram of a diaphragm position at a trigger point and estimated diaphragm motion during an imaging phase in accordance with at least one example embodiment.

FIG. 11 is an image acquired during the imaging phase of FIG. 10 in accordance with at least one example embodiment.

### DETAILED DESCRIPTION OF EXAMPLE EMBODIMENTS

Independent of the grammatical term usage, individuals with male, female or other gender identities are included within the term.

Some detailed example embodiments are disclosed herein. However, specific structural and functional details disclosed herein are merely representative for purposes of describing some example embodiments. Example embodiments may, however, be embodied in many alternate forms and should not be construed as limited to only example embodiments set forth herein.

Accordingly, while example embodiments are capable of various modifications and alternative forms, example embodiments thereof are shown by way of example in the drawings and will herein be described in detail. It should be understood, however, that there is no intent to limit an example embodiment to the particular forms disclosed, but to the contrary, example embodiments are to cover all modifications, combinations, equivalents, and alternatives falling within the scope of an example embodiment. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiment.

The terminology used herein is for the purpose of describing various example embodiment only and is not intended to be limiting of example embodiment. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements and/or groups thereof.

When the words “about” and “substantially” are used in this specification in connection with a numerical value, it is intended that the associated numerical value include a tolerance of +10% around the stated numerical value, unless otherwise explicitly defined. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., +10%) around the stated numerical values or shapes.

Unless otherwise defined, all terms (including technical and scientific terms) used herein have the same meaning as commonly understood by one of ordinary skill in the art to which example embodiment belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

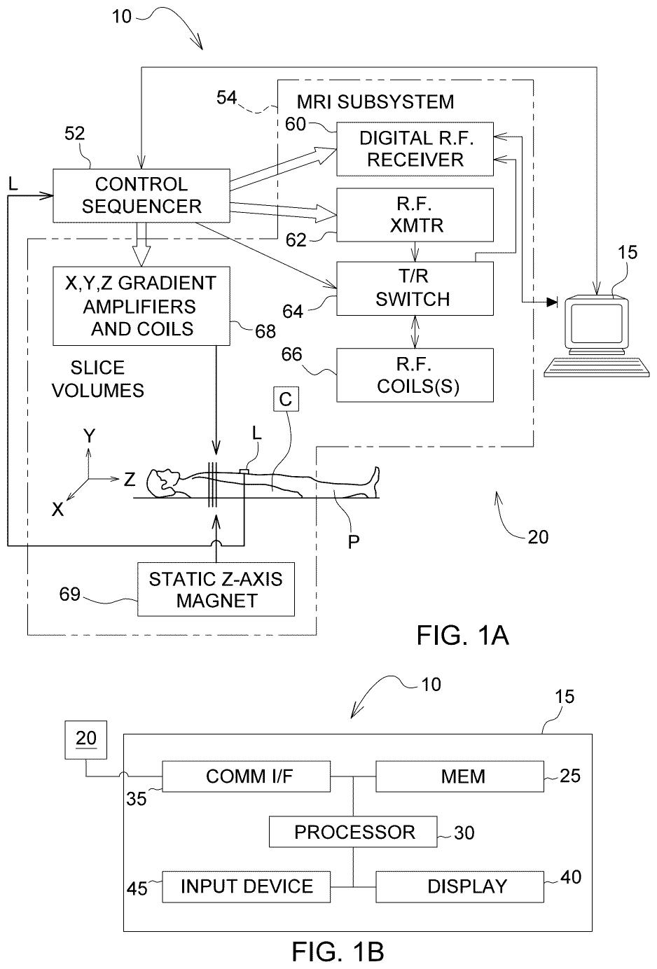

FIG. 1A is an illustration of a system for implementing methods according to example embodiments described herein. FIG. 1B is a block diagram illustrating an example embodiment of the system shown in FIG. 1A. Although one or more example embodiments may be described herein with regard to the systems shown in FIGS. 1A and 1B, example embodiments should not be limited to these examples.

Referring to FIGS. 1A and 1B, a system 10 may include an information processing device 15 and an acquisition device 20. The acquisition device 20 includes a magnetic resonance imaging (“MRI”) real-time control sequencer 52 and an MRI subsystem 54. The MRI subsystem 54 may include XYZ magnetic gradient coils and associated amplifiers 68, a static Z-axis magnet 69, a digital radiofrequency (“RF”) transmitter 62, a digital RF receiver 60, a transmit/receive switch 64, and RF coil(s) 66. The acquisition device 20 may include additional or fewer components in some example embodiments, and may be configured to image a patient.

The MRI subsystem 54 may be controlled in real-time by the MRI real-time control sequencer 52 to generate and measure magnetic field and radio frequency emissions that stimulate nuclear magnetic resonance (“NMR”) phenomena in an object P (e.g., a human or other living body) to be imaged.

The information processing device 15 may implement a method for processing medical data, such as medical image data. As discussed in more detail below, one or more information processing devices such as the information processing device 15 may be configured to implement any or all of the example embodiments described herein.

In FIGS. 1A and 1B, the acquisition device 20 is shown as a separate unit from the information processing device 15. It is, however, possible to integrate the information processing device 15 as part of the acquisition device 20.

The information processing device 15 may include at least one memory 25, processing circuitry including at least one processor 30, at least one communication interface 35 and/or an input device 40. The at least one memory 25 may include various special purpose program code including computer executable instructions which may cause the at least one processor 30 of the information processing device 15 to perform one or more of the methods according to example embodiments described herein. The acquisition device 20 may provide the medical data to the information processing device 15 via the input device 40. In some example embodiments, the information processing device 15 may additionally include a display 45 that may be configured to output information about one or more of an imaging process, the information processing device 15, or the acquisition device 20.

As will be appreciated, depending on the implementation of the system 10, the system 10 may include additional components. However, it is not necessary that all of these generally conventional components be shown in order to disclose the illustrative example embodiment. For example purposes, the system 10 will be discussed with regard to the at least one processor 30. However, it should be understood that the system 10 may include one or more processors or other processing circuitry, such as one or more Application Specific Integrated Circuits (“ASICs). The at least one processor 30 may include, but is not limited to, a central processing unit (“CPU”), an arithmetic logic unit (“ALU”), a graphics processing unit (“GPU”), an application processor (“AP”), a digital signal processor (“DSP”), a microcomputer, a field programmable gate array (“FPGA”), and programmable logic unit, ASIC, a neural network processing unit (“NPU”), an Electronic Control Unit (“ECU”), a quantum computer, and the like. In some example embodiments, the processing circuitry may include a non-transitory computer readable storage medium or device (e.g., memory), for example a solid state drive (“SSD”), storing a program of instructions, and a processor (e.g., CPU) configured to execute the program of instructions to implement the functionality and/or methods performed by some or all of the systems according to any of the example embodiments.

The at least one memory 25 may be a computer readable storage medium that generally includes a random access memory (“RAM”), read only memory (“ROM”), and/or a permanent mass storage device, such as a disk drive. The at least one memory 25 may also store an operating system and any other routines/modules/applications for providing the functionalities of the system 10 to be executed by the at least one processor 30. These software components may also be loaded from a separate computer readable storage medium into the at least one using a drive mechanism (not shown). Such separate computer readable storage medium may include a disc, tape, DVD/CD-ROM drive, memory card, or other like computer readable storage medium (not shown). In some example embodiments, software components may be loaded into the at least one memory 25 via one of the at least one communication interface 35, rather than via a computer readable storage medium.

The at least one processor 30 or other processing circuitry may be configured to carry out instructions of a computer program by performing the arithmetical, logical, and input/output operations of the system. Instructions may be provided to the at least one processor 30 by the at least one memory 25.

The at least one communication interface 35 may be wired and may include components that interface the at least one processor 30 with the other input/output components. As will be understood, the at least one communication interface 35 and programs stored in the at least one memory 25 to set forth the special purpose functionalities of the system 10 will vary depending on the implementation of the system 10.

The at least one communication interface 35 may also include one or more user input devices (e.g., a keyboard, a keypad, a mouse, or the like) and user output devices (e.g., a display, a speaker, or the like).

As disclosed herein, the term “storage medium,” “computer readable storage medium” or “non-transitory computer readable storage medium” may represent one or more devices for storing data, including ROM, RAM, magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing or carrying instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store a computer program or computer program code, and the at least one memory and the computer program code may be configured to, with at least one processor, the methods described herein. Additionally, the processor, memory and example algorithms, encoded as computer program code, serve as means for providing or causing performance of operations discussed herein. At least one other example embodiment may include a computer program including program segments or instructions that, when executed by at least one processor of a system, cause the system to perform the functions and methods described herein.

A code segment of a computer program may represent a procedure, function, subprogram, program, routine, subroutine, module, software package, class, or any combination of instructions, data structures or program statements. A code segment may be coupled to another code segment or a hardware circuit by passing and/or receiving information, data, arguments, parameters or memory contents. Information, arguments, parameters, data, etc. may be passed, forwarded, or transmitted via any suitable technique including memory sharing, message passing, token passing, network transmission, etc.

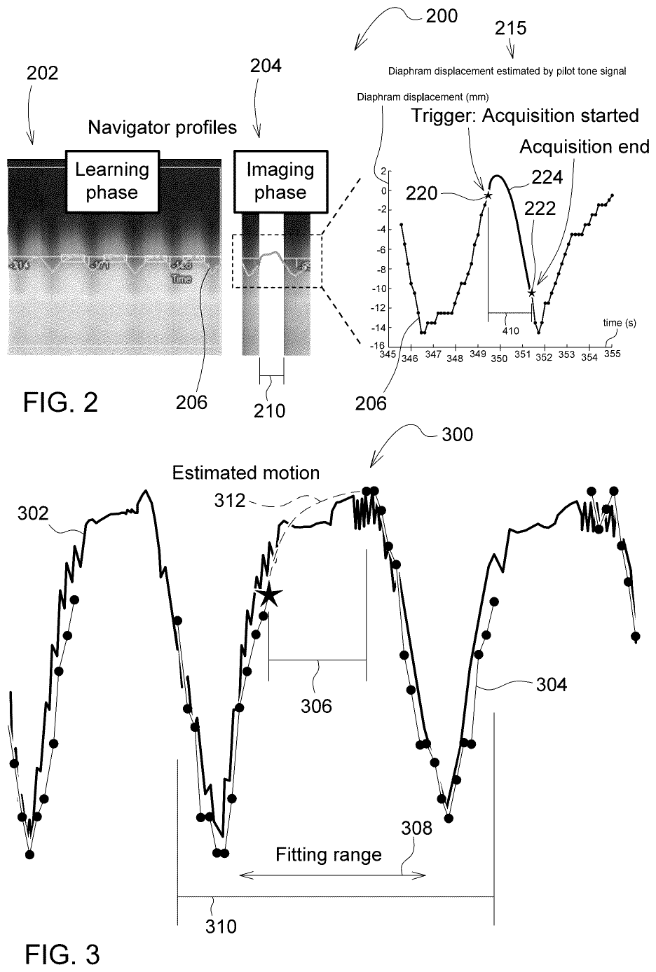

FIGS. 2-7 will be discussed herein with respect to the system 10 of FIGS. 1A and 1B. Example embodiments should not, however, be limited to this example.

Navigator triggering is commonly used in various MR imaging applications. For example, navigator triggering is common in free-breathing magnetic resonance cholangiopancreatography (“MRCP”). Navigators are RF pulses that are used to dynamically track anatomic motion, in particular, motion at or around the diaphragm. Typically, MR imaging via navigator triggering involves a learning phase and an imaging phase. In a learning phase, navigator pulses are applied in each repetition time (“TR”) of a plurality of TRs which enables a computer system such as the system 10 or a user to determine an acquisition window. The acquisition window is determined based on diaphragm motion that is acquired during the learning phase. The diaphragm motion provides information about respiratory patterns, breathing rate, and average end-inspiration and end-expiration positions to be determined which are used to determine the acquisition window. In the imaging phase, an imaging region or target region is imaged during the acquisition window determined in the learning phase.

While the navigator is configured to track an absolute motion of the diaphragm during the learning phase and portions of the imaging phase, it is unable to track the absolute motion of the diaphragm during the acquisition window of the imaging phase. Thus, absolute movement of the diaphragm is unknown during this time period. Systems and methods described herein provide improved imaging methods by providing an estimation of diaphragm motion during the acquisition window of the imaging phase. The estimation of the diaphragm motion may also be used to predict an image quality and/or correct for motion artifacts in resultant images acquired during the imaging phase.

FIG. 2 is a diagram 200 illustrating diaphragm motion during a learning phase 202 and an imaging phase 204 of an MR imaging acquisition using navigator triggering, according to an example embodiment. During the learning phase, a position of the diaphragm 206 is continuously measured. The position of the diaphragm 206 is shown with respect to time during the learning phase 202 and the imaging phase 204.

The imaging phase 204 includes a gap 210. The gap 210 is a time period during which the position of the diaphragm is unable to be measured by the navigator. The gap 210 corresponds to an acquisition window as described above.

Graph 215 illustrates a portion of the imaging phase 204. The x-axis of the graph 215 represents time in seconds and the y-axis of the graph 215 represents diaphragm displacement in millimeters. The position of the diaphragm 206 is measured until a first point 220 and is then measured again starting at a second point 222. A time between the first point 220 and the second point 222 may be the acquisition window. A measured diaphragm position 224 is shown between the first point 220 and the second point 222. The measured diaphragm position 224 may be determined as described in further detail herein.

In at least one example embodiment, the navigator signal and one or more additional signals may be used to estimate diaphragm motion during the acquisition window. The one or more additional signals may be signals from one or more sensors including at least one of respiratory bellows, biomatrix respiratory sensors, or a pilot tone. The one or more sensors may be configured to sense diaphragm motion by sensing an exterior of a patient or subject during an imaging procedure. The one or more sensors may provide a continuous measurement during a duration of both the learning phase and the imaging phase as described above.

In at least one example embodiment, a correlation model may be determined. The correlation model describes a relationship between the navigator signals and signals from the one or more sensors. In at least one example embodiment, the correlation model may be determined during the learning phase when both the navigator signal and signals from the one or more sensors are continuously acquired. During the imaging phase, the correlation model may be updated or re-tuned as additional signals are acquired from the navigator and the one or more sensors. By updating the correlation model (e.g., continuously), the correlation model may be re-tuned every trigger cycle to avoid potential offset errors, for instance, those arising from RF interference for pilot tone signal processing.

The one or more sensors may acquire signals continuously throughout the imaging phase, but the navigator may only acquire signals outside of the acquisition window as described above. The correlation model may be used to estimate diaphragm motion during the acquisition window. The estimated diaphragm motion may be used to correct for motion artifacts in an image obtained during the acquisition window of the imaging phase and/or to predict an overall image quality before the acquisition window of the imaging phase is complete.

In at least one example embodiment, the correlation model may be a quadratic function that relates a high temporal resolution respiratory signal with low temporal resolution diaphragm motion data. The high temporal resolution respiratory signal may be the navigator signal and the low temporal resolution diaphragm motion data may be acquired from the one or more sensors. In at least one example embodiment, the correlation model may be given by Equation (1) shown below:

y_{SI}=φ(s)=as²+bs+c+d\dot{s}+e\dot{s}² (1)

In Equation (1), a, b, c, d, and e are constants determined from the learning phase, s is the high temporal resolution respiratory signal, \dot{s} is a derivative of the high temporal resolution respiratory signal, and y_{SI} is the low temporal resolution diaphragm motion data.

FIG. 3 is diagram 300 illustrating an example of a surrogate signal of respiratory motion from a sensor and estimated diaphragm motion during an imaging phase in accordance with at least one example embodiment. The surrogate signal of respiratory motion from the sensor may be obtained from one or more of the sensors as described above. In at least one example embodiment, the imaging phase as illustrated in FIG. 3 may be a time period when the correlation model is updated or re-tuned.

Referring to FIG. 3, a first type of respiratory signals 302 may be acquired from the one or more external sensors. The first type of respiratory signals 302 may be continuous as shown in FIG. 3. A second type of respiratory signals 304 may be the navigator signal that is acquired during the imaging phase other than during an acquisition window 306.

In at least one example embodiment, the correlation model may be re-tuned by synchronizing the first type of respiratory signals 302 and the second type of respiratory signals 304 during a fitting range 308 except during the acquisition window 306 when the second type of respiratory signals 304 are not acquired. Respiratory motion or breathing may be classified as normal or fast in at least one example embodiment. For example, normal breathing may include deep breathing and fast breathing may involve and inhale and exhale of a patient occurring in less time than in the normal or deep breathing situation. Normal breathing may be about 12-20 breaths per minute for an adult and fast breathing may be a breathing rate of more than 20 breaths per minute. Deep breathing may have a breathing rate within the normal breathing range with a larger amplitude than normal breathing that is not deep breathing. A breathing rate and an amplitude of breaths may vary more in a fast breathing scenario than in a normal breathing scenario. In a fast breathing situation, the fitting range 308 may be expanded to an expanded fitting range 310 to avoid underfitting of the correlation model due to lack of data.

After being re-tuned, the correlation model is applied to estimate diaphragm motion during the acquisition window 306. The dashed line 312 in FIG. 3 represents the estimated diaphragm motion that is determined from the correlation model and the first type of respiratory signals 302 during the acquisition window 306.

After an estimated diaphragm motion is determined from the correlation model, the estimated diaphragm motion may be processed by applying a filter to the results. In at least one example embodiment, the filter may be a Kalman filter. In at least one example embodiment, a procession model may be associated with the Kalman filter. The procession model may be established for respiratory motion and may be initialized using a periodic motion model (PM(ω) Model) given by Equation (2) shown below.

$$PM ( ω ) Model \{ \begin{matrix}{x}_{k} = ( \begin{matrix}{X}_{k} \\ {V}_{k}\end{matrix} ) = ( \begin{matrix}1 & Δ t \\ - {ω}^{2} Δ t & 1\end{matrix} ) {x}_{k - 1} + {w}_{k} \\ {z}_{k} = ( \begin{matrix}1 & 0\end{matrix} ) {x}_{k} + {v}_{k}\end{matrix} \qquad ( 2 )$$

$$with \{ \begin{matrix}{w}_{k} ∼ N ( 0 , {Q}_{k} ) \\ {v}_{k} ∼ N ( 0 , {R}_{k} )\end{matrix}$$

In Equation (2), x is a state variable of the procession model that is a vector including displacement (X) and velocity (V), k is an index of a discrete time series of the periodic motion model, ω is a frequency of respiratory motion, Δt is a temporal resolution of the estimated diaphragm motion results, w and v are Gaussian random variables with zero mean, Q and R are covariance matrices of uncertainties in the periodic motion model, and N is a Gaussian normal distribution. Thus, the periodic motion model is Gaussian with a model uncertainty with a mean of zero and covariance matrices Q and R represented by N (0, Qₖ) and N(0, Rₖ). The Kalman filter may be configured to take a noisy measurement z as an input to estimate the state variable x.

In at least one example embodiment, the procession model may be trained using a non-supervised learning model. For example, an expectation maximization may be used to train the parameters of the procession model for the Kalman filter during the learning phase of the MR imaging acquisition using navigator triggering.

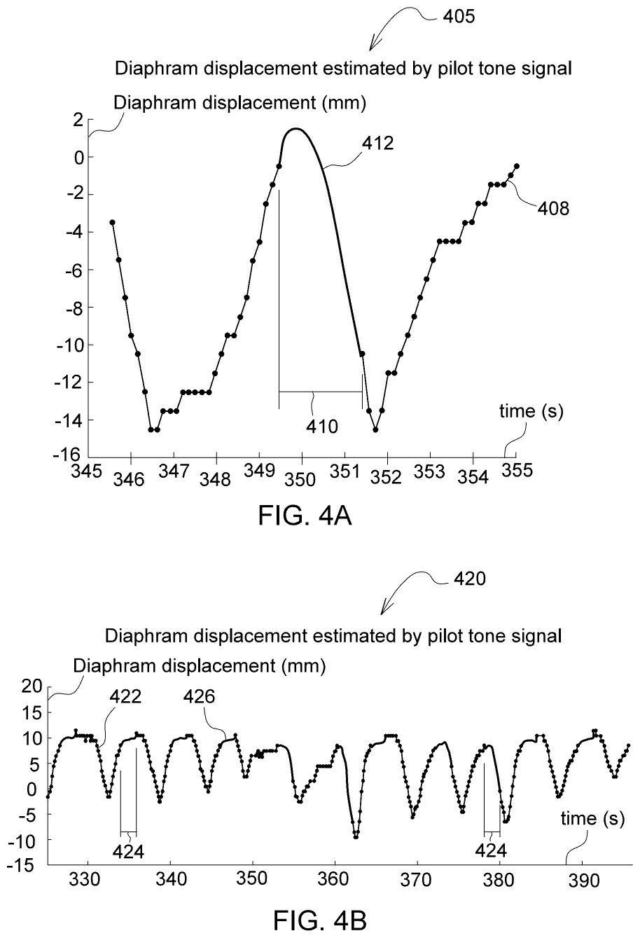

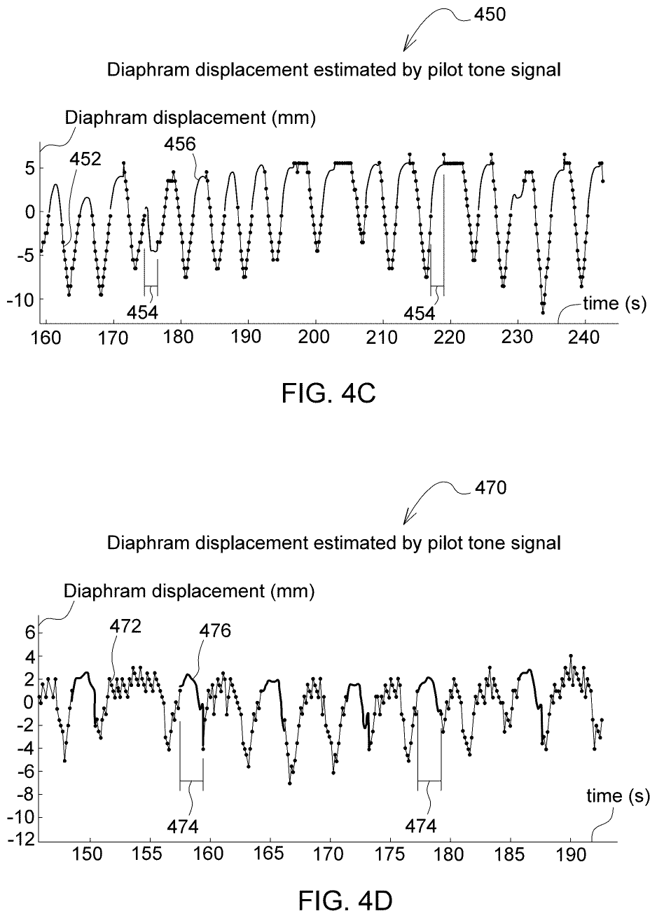

FIGS. 4A-4D illustrate example embodiments of diaphragm motion estimated using a correlation model as described above. In at least one example embodiment, the one or more sensors may be a pilot tone and the signal from the one or more sensors may be a pilot tone signal that is used to estimate the diaphragm motion during the acquisition window. As shown in each of the graphs of FIGS. 4A-4D, the x-axis represents time in seconds(s) and the y-axis represents diaphragm displacement in millimeters (mm). The signals illustrated in FIGS. 4A-4D were acquired from patients or subjects on 1.5 T and/or 3 T scanners and a navigator-triggered 3D T2 SPACE sequence was applied using Beat Sensor integrated BioMatrix body coil to generate and store pilot tone signals in the scan data.

FIG. 4A illustrates a graph 405 with a navigator signal 408 illustrating diaphragm motion other than during an acquisition window 410. The correlation model is used to determine an estimated diaphragm motion 412 during the acquisition window 410.

FIG. 4B illustrates a graph 420 with a navigator signal 422 illustrating diaphragm motion other than during a plurality of acquisition windows 424. The correlation model is used to determine an estimated diaphragm motion 426 during each acquisition window of the plurality of acquisition windows 424.

FIG. 4C illustrates a graph 450 with a navigator signal 452 illustrating diaphragm motion other than during a plurality of acquisition windows 454. The correlation model is used to determine an estimated diaphragm motion 456 during each acquisition window of the plurality of acquisition windows 454.

FIG. 4D illustrates a graph 470 with a navigator signal 472 illustrating diaphragm motion other than during a plurality of acquisition windows 474. The correlation model is used to determine an estimated diaphragm motion 476 during each acquisition window of the plurality of acquisition windows 474.

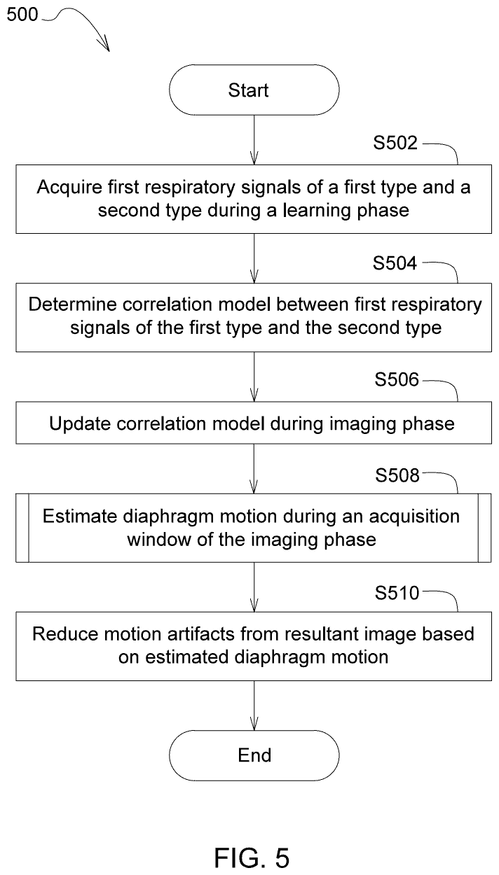

FIG. 5 illustrates a method 500 for reducing motion artifacts from a resultant image based on estimated diaphragm motion in accordance with at least one example embodiment.

Referring to FIG. 5, the method 500 begins at S502 where first respiratory signals of a first type and first respiratory signals of a second type are acquired. The first respiratory signals may be acquired during a learning phase. In at least one example embodiment, the first type of signals may be signals acquired from the one or more external sensors as described above and the second type of signals may be the navigator signals as described above. Both the first type and second type of signals may be acquired continuously during the learning phase as described above.

At S504, the at least one processor 30 may determine a correlation model between the first respiratory signals of the first type and the first respiratory signals of the second type. The correlation model may be the correlation model described above.

At S506, the at least one processor 30 may update the correlation model during an imaging phase. As described above, the correlation model may be re-tuned during the imaging phase other than during an acquisition window of the imaging phase.

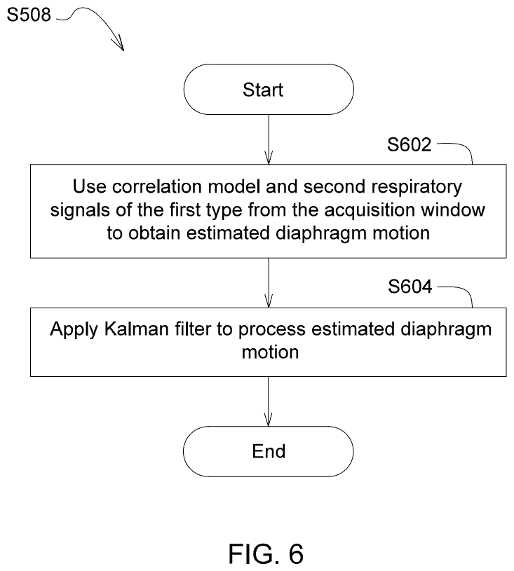

At S508, the at least one processor 30 may estimate diaphragm motion during the acquisition window of the imaging phase. An example embodiment of S508 will be described below with respect to FIG. 6.

At S510, the at least one processor 30 may reduce motion artifacts in resultant images from the imaging phase based on the estimated diaphragm motion during the acquisition window. In at least one example embodiment, motion artifacts may be reduced by using the estimated diaphragm data to perform additional k-space data binning, data rejection and reacquisition, and/or phase correction in resultant images. For example, from the estimated diaphragm data, motion vector fields may be estimated within a field of view of the resultant images. The motion vector fields may be applied to the image data to correct k-space data during image reconstructions.

FIG. 6 illustrates an example embodiment of a method of estimating diaphragm motion at S508 in FIG. 5.

Referring to FIG. 6, at S602, the at least one processor 30 may use the correlation model and second respiratory signals of the first type from the acquisition window to estimate diaphragm motion during the acquisition window. As described above, the acquisition window is the portion of the imaging phase where the navigator signals are not obtained and where diaphragm motion cannot be directly measured. Thus, the correlation model and signals from the one or more sensors are used to obtain estimated diaphragm motion during the acquisition window.

At S604, the at least one processor 30 may apply a filter, such as a Kalman filter, to the estimated diaphragm motion to process the estimated diaphragm motion. For example, respiratory motion may be smoothed by applying the Kalman filter to the estimated diaphragm motion.

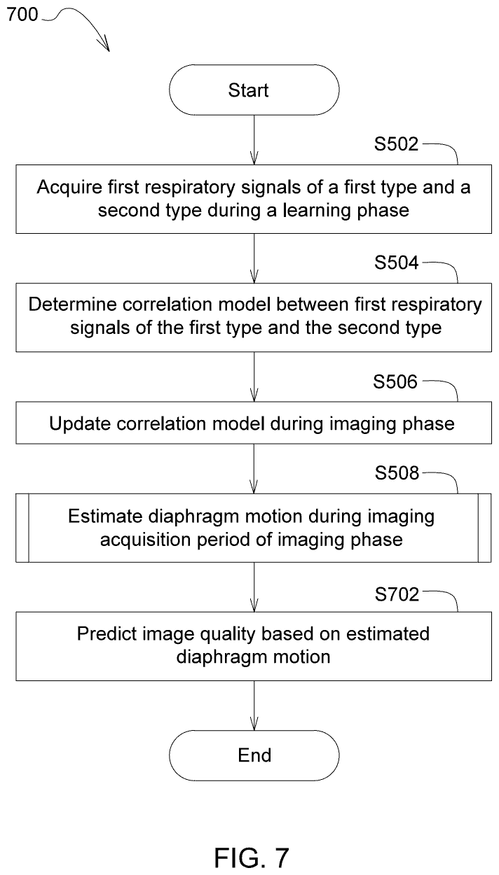

FIG. 7 illustrates a method 700 for predicting image quality based on estimated diaphragm motion in accordance with at least one example embodiment.

Referring to FIG. 7, the method 700 may include S502-S508 as described above with respect to FIG. 5.

At S702, the at least one processor 30 may be configured to predict an image quality of resultant images from the acquisition window of the imaging phase based on the estimated diaphragm motion determined at S508. For example, if the estimated diaphragm motion shows irregular diaphragm motion, an image quality may be predicted as being lower than an image quality of images where diaphragm motion was consistent with a previous TR or consistent with diaphragm motion in the learning phase of the MR image acquisition. In at least one example embodiment, understanding an image quality before imaging of a target region is complete may result in improved imaging methods by providing more information related to the resultant images before they are output by the system 10. The systems and methods described herein may be used with free breathing magnetic resonance cholangiopancreatography (MRCP), which generally has a relatively long acquisition time. Due to the relatively long acquisition time, it may be burdensome to repeat an entire scan if an operator such as an MR technologist determined that the image quality is not sufficient. Therefore, predicting an image quality before the scan is complete may allow the operator to pause and/or stop the scan to reinstruct the patient to breath normally to improve final image results. Thus, total scan time may be reduced because an entire scan may not need to be repeated.

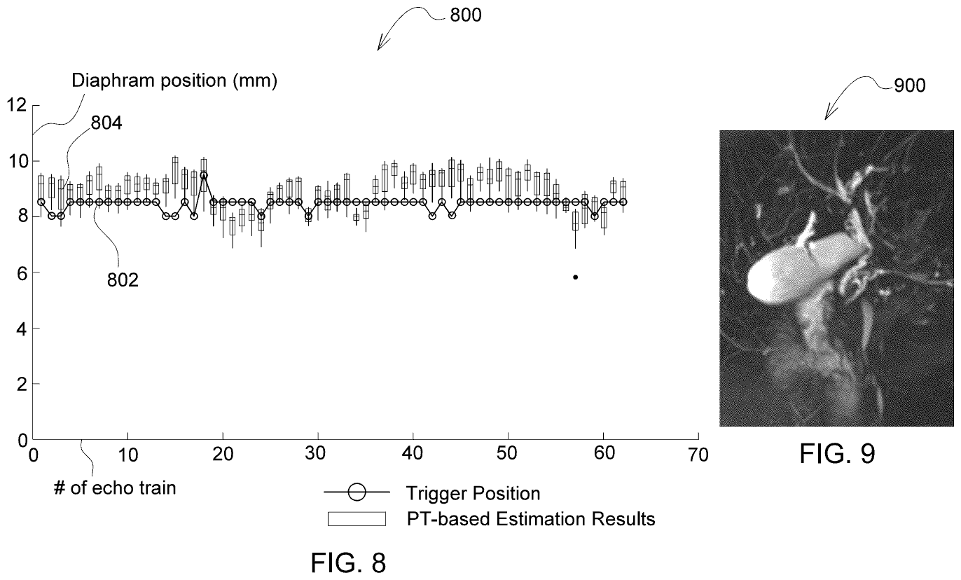

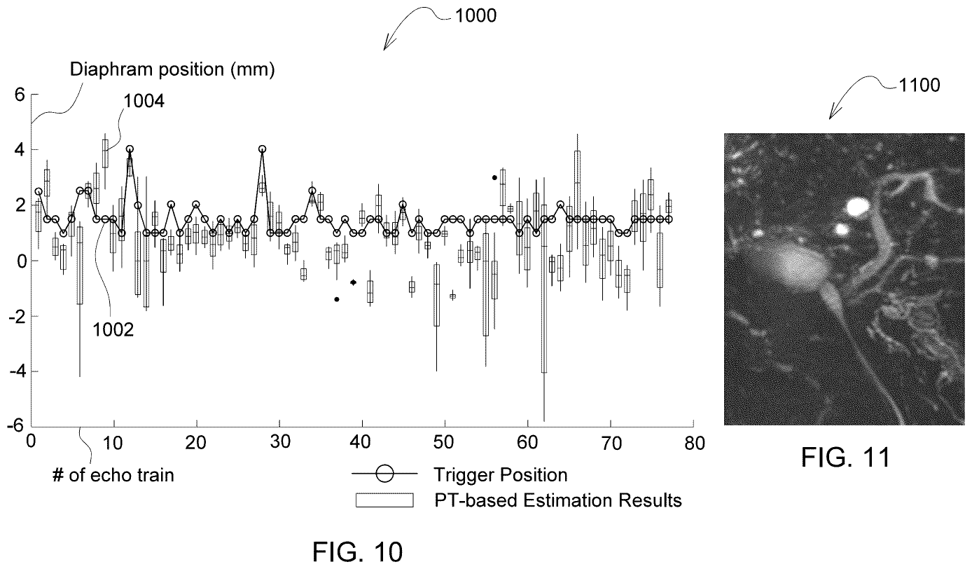

FIGS. 8-11 show an example relationship between resultant images and a diaphragm position at a trigger point and estimated diaphragm motion during an imaging phase. In particular, FIGS. 8 and 10 are diagrams of a diaphragm position at a trigger point and estimated diaphragm motion during each imaging phase and FIGS. 9 and 11 are images acquired during the imaging phases of FIGS. 8 and 10, respectively. The trigger point may be a point in time defining a beginning of an acquisition window such as the first point 220 described above in FIG. 2.

Referring to FIG. 8, graph 800 illustrates a diaphragm position at a trigger point 802 and estimated diaphragm motion 804 during each imaging phase. In particular, the graph 800 includes an x-axis representing a number of echoes and a y-axis representing diaphragm position. As shown in the graph 800, both the diaphragm position at the trigger point 802 and the estimated diaphragm motion 804 are relatively consistent between adjacent echoes without any large fluctuations. Further, the discrepancies between the estimated diaphragm motion 804 and the diaphragm position at the trigger point 802 are relatively small. Thus, an image 900 of FIG. 9 appears clear and does not appear to include large motion artifacts.

Referring to FIG. 10, graph 1000 illustrates a diaphragm position at a trigger point 1002 and estimated diaphragm motion 1004 during each imaging phase. In particular, the graph 1000 includes an x-axis representing a number of echoes and a y-axis representing diaphragm position. As shown in the graph 1000, both the diaphragm position at the trigger point 1002 and the estimated diaphragm motion 1004 are inconsistent between echoes and both include large fluctuations of diaphragm motion between adjacent echoes. Further, the discrepancies between the estimated diaphragm motion 1004 and the diaphragm position at the trigger point 1002 are relatively large. Thus, an image 1100 of FIG. 11 is blurry and includes motion artifacts that impact the image quality.

In practice, an operator may have stopped or paused the scan occurring during FIGS. 10 and 11 to reinstruct a patient as described above. Based on the diaphragm position at the trigger point 1002 and the estimated diaphragm motion 1004, image quality may have been predicted as containing impactful motion artifacts that may require further scanning in order to obtain desirable results.

The above-described systems and methods provide methods of estimating diaphragm motion. The systems and methods described herein may provide improved MR imaging methods that reduce or eliminate the need to have patients or subjects hold their breath during an image acquisition because the systems and methods may be used to estimate diaphragm motion that can be used to correct motion artifacts in resultant images. Thus, the systems and methods described herein may improve motion robustness and overall images quality of navigator triggered sequences by providing continuous motion information during an image acquisition.

In particular, diaphragm motion as determined from a navigator signal may be estimated during an acquisition window by applying the systems and methods described herein. Understanding diaphragm motion during an acquisition window of an imaging phase of an MR imaging procedure may result in improved imaging procedures and improved methods for predicting imaging quality. In particular, estimated diaphragm motion may be used to both predict an image quality before the imaging phase is complete and correct motion artifacts in resultant images from the imaging phase.

Example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

### NON-LIMITING ILLUSTRATIVE EMBODIMENTS

The following is a list of non-limiting illustrative embodiments disclosed herein:

Illustrative embodiment 1 includes a system comprising: at least one memory configured to store instructions; and at least one processor configured to execute the instructions to cause the system to acquire, during a first phase, first respiratory signals of a first type and first respiratory signals of a second type, determine a correlation model correlating the first respiratory signals of the first type and the first respiratory signals of the second type, update the correlation model based on second respiratory signals of the first type and second respiratory signals of the second type, acquired during a second phase, the second phase including an imaging acquisition period during which the second respiratory signals of the first type are acquired, and estimate diaphragm motion based on the second respiratory signals of the first type and the correlation model.

Illustrative embodiment 2 includes the system of illustrative embodiment 1, wherein the at least one processor is further configured to cause the system to process the diaphragm motion with a filter.

Illustrative embodiment 3 includes the system of illustrative embodiment 2, wherein the filter is a Kalman filter and a procession model of the Kalman filter is trained using a non-supervised learning method.

Illustrative embodiment 4 includes the system of any one of the preceding illustrative embodiments, wherein the correlation model is a quadratic function defining a relationship between the first respiratory signals of the first type and the first respiratory signals of the second type.

Illustrative embodiment 5 includes the system of any one of the preceding illustrative embodiments, wherein the second respiratory signals of the second type are generated from a navigator-triggered sequence and the second respiratory signals of the second type are not acquired during the imaging acquisition period.

Illustrative embodiment 6 includes the system of any one of the preceding illustrative embodiments, wherein the first phase is a training phase and the second phase is an imaging phase.

Illustrative embodiment 7 includes the system of any one of the preceding illustrative embodiments, wherein the diaphragm motion is an estimation of the second respiratory signals of the second type during the imaging acquisition period.

Illustrative embodiment 8 includes the system of any one of the preceding illustrative embodiments, wherein the at least one processor is further configured to cause the system to correct for motion artifacts in an image obtained during the imaging acquisition period based on the diaphragm motion.

Illustrative embodiment 9 includes the system of any one of the preceding illustrative embodiments, wherein the at least one processor is further configured to cause the system to predict an overall image quality before the imaging acquisition period is complete.

Illustrative embodiment 10 includes the system of any one of the preceding illustrative embodiments, wherein the first respiratory signals of the first type and the second respiratory signals of the first type are acquired by one or more sensors, the one or more sensors including at least one of respiratory bellows, biomatrix respiratory sensors, or a pilot tone.

Illustrative embodiment 11 includes a method comprising: acquiring, during a first phase, first respiratory signals of a first type and first respiratory signals of a second type; determining a correlation model correlating the first respiratory signals of the first type and the first respiratory signals of the second type; updating the correlation model based on second respiratory signals of the first type and second respiratory signals of the second type, acquired during a second phase, the second phase including an imaging acquisition period during which the second respiratory signals of the first type are acquired; and estimating diaphragm motion based on the second respiratory signals of the first type and the correlation model.

Illustrative embodiment 12 includes the method of illustrative embodiment 11, further comprising: processing the diaphragm motion with a filter.

Illustrative embodiment 13 includes the method of illustrative embodiment 12, wherein the filter is a Kalman filter and a procession model of the Kalman filter is trained using a non-supervised learning method.

Illustrative embodiment 14 includes the method of any one of illustrative embodiments 11-13, wherein the correlation model is a quadratic function defining a relationship between the first respiratory signals of the first type and the first respiratory signals of the second type.

Illustrative embodiment 15 includes the method of any one of illustrative embodiments 11-14, wherein the second respiratory signals of the second type are generated from a navigator-triggered sequence and the second respiratory signals of the second type are not acquired during the imaging acquisition period.

Illustrative embodiment 16 includes the method of any one of illustrative embodiments 11-15, wherein the first phase is a training phase and the second phase is an imaging phase.

Illustrative embodiment 17 includes the method of any one of illustrative embodiments 11-16, wherein the diaphragm motion is an estimation of the second respiratory signals of the second type during the imaging acquisition period.

Illustrative embodiment 18 includes the method of any one of illustrative embodiments 11-17, further comprising: correcting for motion artifacts in an image obtained during the imaging acquisition period based on the diaphragm motion.

Illustrative embodiment 19 includes the method of any one of illustrative embodiments 11-18, further comprising: predicting an overall image quality before the imaging acquisition period is complete.

Illustrative embodiment 20 includes the method of any one of illustrative embodiments 11-19, wherein the first respiratory signals of the first type and the second respiratory signals of the first type are acquired by one or more sensors, the one or more sensors including at least one of respiratory bellows, biomatrix respiratory sensors, or a pilot tone.

We claim:

1. A system comprising:
at least one memory configured to store instructions; and
at least one processor configured to execute the instructions to cause the system to
acquire, during a first phase, first respiratory signals of a first type and first respiratory signals of a second type,
determine a correlation model correlating the first respiratory signals of the first type and the first respiratory signals of the second type,
update the correlation model based on second respiratory signals of the first type and second respiratory signals of the second type, acquired during a second phase, the second phase including an imaging acquisition period during which the second respiratory signals of the first type are acquired, and
estimate diaphragm motion based on the second respiratory signals of the first type and the correlation model.

2. The system of claim 1, wherein the at least one processor is further configured to cause the system to process the diaphragm motion with a filter.

3. The system of claim 2, wherein the filter is a Kalman filter and a procession model of the Kalman filter is trained using a non-supervised learning method.

4. The system of claim 1, wherein the correlation model is a quadratic function defining a relationship between the first respiratory signals of the first type and the first respiratory signals of the second type.

5. The system of claim 1, wherein the second respiratory signals of the second type are generated from a navigator-triggered sequence and the second respiratory signals of the second type are not acquired during the imaging acquisition period.

6. The system of claim 1, wherein the first phase is a training phase and the second phase is an imaging phase.

7. The system of claim 1, wherein the diaphragm motion is an estimation of the second respiratory signals of the second type during the imaging acquisition period.

8. The system of claim 1, wherein the at least one processor is further configured to cause the system to correct for motion artifacts in an image obtained during the imaging acquisition period based on the diaphragm motion.

9. The system of claim 1, wherein the at least one processor is further configured to cause the system to predict an overall image quality before the imaging acquisition period is complete.

10. The system of claim 1, wherein the first respiratory signals of the first type and the second respiratory signals of the first type are acquired by one or more sensors, the one or more sensors including at least one of respiratory bellows, biomatrix respiratory sensors, or a pilot tone.

11. A method comprising:
acquiring, during a first phase, first respiratory signals of a first type and first respiratory signals of a second type;
determining a correlation model correlating the first respiratory signals of the first type and the first respiratory signals of the second type;
updating the correlation model based on second respiratory signals of the first type and second respiratory signals of the second type, acquired during a second phase, the second phase including an imaging acquisition period during which the second respiratory signals of the first type are acquired; and
estimating diaphragm motion based on the second respiratory signals of the first type and the correlation model.

12. The method of claim 11, further comprising:
processing the diaphragm motion with a filter.

13. The method of claim 12, wherein the filter is a Kalman filter and a procession model of the Kalman filter is trained using a non-supervised learning method.

14. The method of claim 11, wherein the correlation model is a quadratic function defining a relationship between the first respiratory signals of the first type and the first respiratory signals of the second type.

15. The method of claim 11, wherein the second respiratory signals of the second type are generated from a navigator-triggered sequence and the second respiratory signals of the second type are not acquired during the imaging acquisition period.

16. The method of claim 11, wherein the first phase is a training phase and the second phase is an imaging phase.

17. The method of claim 11, wherein the diaphragm motion is an estimation of the second respiratory signals of the second type during the imaging acquisition period.

18. The method of claim 11, further comprising:
correcting for motion artifacts in an image obtained during the imaging acquisition period based on the diaphragm motion.

19. The method of claim 11, further comprising:
predicting an overall image quality before the imaging acquisition period is complete.

20. The method of claim 11, wherein the first respiratory signals of the first type and the second respiratory signals of the first type are acquired by one or more sensors, the one or more sensors including at least one of respiratory bellows, biomatrix respiratory sensors, or a pilot tone.
